(19) United States
(12) Patent Application Publication
(10) Pub. No.: US 2024/0293673 A1
(43) Pub. Date: Sep. 5, 2024

(54) NEUROSTIMULATOR TO AID IN RECOVERY FROM STROKE OR OTHER LIMITATIONS IN HUMAN FUNCTION

(71) Applicant: Cortigent, Inc., Sylmar, CA (US)

(72) Inventors: Proyag Datta, Thousand Oaks, CA (US); Jerry Ok, Canyon Country, CA (US); Uday Patel, Los Angeles, CA (US)

(73) Assignee: Cortigent, Inc., Sylmar, CA (US)

(21) Appl. No.: 18/117,299

(22) Filed: Mar. 3, 2023

(51) Int. Cl. A61N 1/36; A61N 1/05; A61N 1/378

(52) U.S. Cl. CPC A61N 1/36103; A61N 1/0531; A61N 1/36139; A61N 1/3787

(57) ABSTRACT

Briefly, and in general terms, the present invention is directed to a cortical stimulator for neuromodulation. The cortical stimulator is configured so as to stimulate or block the operation of a portion of the cortex, for example, but not limited to, the motor cortex of a subject who has suffered a stroke. The present disclosure of a cortical stimulator includes a flexible circuit electrode array connected to an electronics package and a coil inductively coupled to an external coil for power and data. The present invention includes an electrode array significantly larger than the area intended to be stimulated. The larger electrode array allows a clinician to select the location of stimulation after completion of the surgery.

### FIELD OF THE INVENTION

The present disclosure relates to cortical neural stimulation. More particularly, it relates to an apparatus, method of use, and method of manufacture for a system including an implantable electrode array, connected electronics package, implantable coil, and associated external coil and support electronics for a cortical stimulator to aid in stroke recovery, and a variety of other conditions.

### BACKGROUND OF THE INVENTION

Approximately 7.6 million Americans over the age of 20 have reported a history of stroke. While rehabilitation therapy may restore some function for many stroke victims, approximately 40% of these patients suffer motor impairments require special care. Consequently, stroke is a leading cause of long-term disability, costing the American economy $38 billion per year due to under-employment and $35 billion in direct healthcare costs. Each year, 610,000 Americans have a first stroke and the age of first stroke is trending younger. This trend portends a growing burden due to under-employment, estimated to reach $56.5 billion per year by 2030.

Rehabilitation therapy after stroke attempts to restore the functions lost due to the patient's stroke. These functions may include movement, speech, strength, and activities of daily living. Conventional rehabilitation therapy for upper extremity function typically utilizes a variety of strategies including strength, balance, stretching, and manual dexterity exercises, and functional task practice of activities of daily living (ADLs) using either the impaired or the less-affected side. Rehabilitation therapy typically follows a course of in-patient rehabilitation followed by out-patient rehabilitation either at a clinic or at home. While most functional gains occur during the acute phase post-stroke, evidence suggests that some degree of functional benefit from therapy can occur beyond 12 months post-stroke.

To enhance the efficacy of stroke rehabilitation for upper extremity function, several technological interventions have been evaluated in clinical trials. Repetitive transcranial magnetic stimulation combined with rehabilitation was explored in the Navigated Inhibitory rTMS to Contralesional Hemisphere (NICHE) trial. Electromyographytriggered neuromuscular stimulation was explored in the Explaining PLastICITy after stroke trial. The effect of virtual reality and video games on upper limb motor capacity during the subacute stage of stroke were evaluated in Effectiveness of Virtual Reality Exercises in STroke Rehabilitation (EVREST). Virtual Reality Training for Upper Extremity in Subacute Stroke (VIRTUES) and a trial by Adie and colleagues. Tele-rehabilitation during the subacute and chronic stages of stroke was investigated by Cramer and colleagues. The Robot Assisted Training for the Upper Limb after Stroke (RATULS) trial investigated the effects of robot-assisted therapy on upper limb motor capacity with participants primarily at the chronic stage. The execution of these trials illustrate the feasibility of integrating technological interventions into rehabilitation therapy; however, all of these trials reported similar benefits to those produced by a matched dose of conventional therapy, recreational activities, or sham stimulation.

Vagus nerve stimulation (VNS) combined with rehabilitation therapy has been shown to improve functional outcomes. VNS activates multiple global neuromodulatory systems throughout the brain. For post-stroke rehabilitation, cholinergic and monoaminergic modulation of motor cortex are hypothesized to enhance the reorganization potential following stroke. Pairing VNS stimulation with rehabilitation (VNS-REHAB) has been shown to elicit a small but clinically meaningful improvement in motor function compared to sham-stimulation controls. This approach recently received FDA approval and is currently available in the market. Still, not all patients respond to VNS-REHAB; 47% achieved a clinically meaningful response versus 24% in controls (rehabilitation therapy only). This leaves a significant number of stroke survivors with a persistent therapeutic need.

Electrical stimulation of the primary motor cortex offers the possibility of much greater target specificity than vagus nerve stimulation. Prior clinical trials combining electrical stimulation of the motor cortex with rehabilitation have been completed with a single channel of stimulation with an effective activation area of 1.8 cm². With continuous stimulation during rehabilitation sessions, this technology demonstrated significant improvements over conventional therapy alone in Phase I and II trials. However, the Everest Phase III trial was unsuccessful in demonstrating efficacy. Combined, these trials established the safety and feasibility of combining cortical stimulation with rehabilitation. The failure of efficacy in Phase III has been attributed, at least in part, to incorrect localization of the stimulating array.

### SUMMARY OF THE INVENTION

Briefly, and in general terms, the present invention is directed to a cortical stimulator for neuromodulation. The cortical stimulator is configured so as to stimulate or block the operation of a portion of the cortex, for example, but not limited to, the motor cortex of a subject who has suffered a stroke.

The present disclosure of a cortical stimulator includes a flexible circuit electrode array connected to an electronics package and a coil inductively coupled to an external coil for power and data. The present invention includes an electrode array significantly larger than the area intended to be stimulated. The larger electrode array allows a clinician to select the location of stimulation after completion of the surgery.

Other features and advantages of the invention will become apparent from the following detailed description, taken in conjunction with the accompanying drawings, which illustrate, by way of example, the features of the invention.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 is a bottom view of the stroke rehabilitation implant.

FIG. 2 is a top perspective view of the stroke rehabilitation implant.

FIG. 3 is a side view of the stroke rehabilitation implant.

FIG. 4 is a view of the stroke rehabilitation implant as implanted on the motor cortex of the brain.

FIG. 5 is a view of an example human brain, including area that can be stimulated to support multiple therapeutic actions.

### DETAILED DESCRIPTION

While general regions of somatotopic representation (i.e. head, arm, leg) in motor cortex as depicted in the homunculus are well accepted, evidence suggests that the somatotopic representation of distinct body segments may be distributed broadly and have multiple representations within the motor cortex. By implanting an array of electrodes over the upper extremity region of the motor cortex, one will be able to map the somatotopic arrangement in unprecedented detail in a controlled, methodical, and reproducible outpatient setting. By recording motor evoked potentials from upper extremity musculature while stimulating individual electrodes or groups of adjacent electrodes, one will be able to generate a motor map that identifies which muscles are activated by suprathreshold stimulation of each electrode or group of electrodes. These motor evoked potentials can serve as a biomarker of physiological response to combined stimulation and rehabilitation. Adjacent electrodes may have functionally distinct motor potentials and may be utilized distinctly during different tasks. The individualized motor maps can be used to determine appropriate stimulation targets for specific tasks. During rehabilitation, these tasks can be practiced while the corresponding stimulation is provided at subthreshold levels. The electrode array can be placed either epidurally or subdurally over the target region of the cortex. To maximize specificity of stimulation, the array should be implanted subdurally.

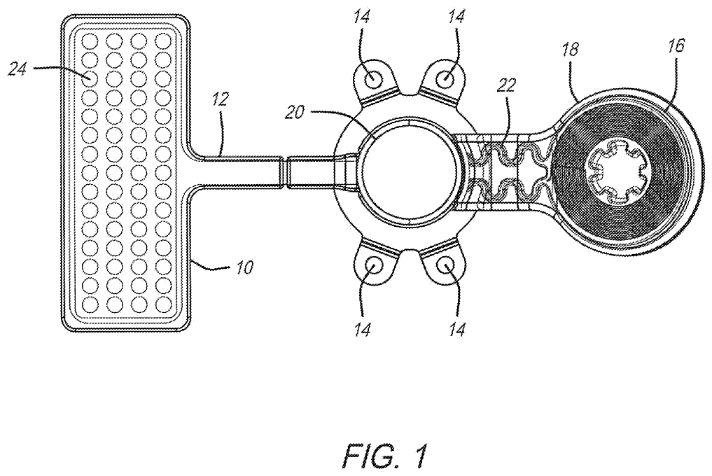

Turning to the drawings FIG. 1 shows a bottom view (the side facing the brain) of the cortical stimulator. Power and data is transferred to the implanted cortical stimulator through an inductive coil 16. The inductive coil is electrically connected in an implantable pulse generator (IPG) within a hermetic package 20 through leads 22. The coil and leads are over molded with a polymer 18, preferably silicone. The hermitic package 20 is adapted to be attached to a skull through voids 14 adapted to accept skull screws (not shown). A flexible circuit electrode array 10 is attached electrically connected to the IPG in the hermetic package 20 by a cable 12. Each electrode 24 has an independent trace leading through the electrode array 10 and cable 12 back to the IPG. It should be noted that the electrode array 10 and cable 12 are formed from a single flexible circuit.

The flexible circuit is preferably a sandwiched polymer body with the electrode array 10 at one end bond pads for connecting the array to the electronics package 20 at the other end, and traces through a cable 12 connecting bond pads to electrodes 24. Polymer materials are useful as electrode array bodies for neural stimulation. They are particularly useful for cortical stimulation for many purposes. Regardless of which polymer is used, the basic construction method is the same. A layer of polymer is laid down, commonly by some form of chemical vapor deposition, spinning, meniscus coating or casting. A layer of metal, preferably platinum, is applied to the polymer and patterned to create electrodes bond pads and leads connecting electrodes to bond pads. Patterning is commonly done by photolithographic methods. A second layer of polymer is applied over the metal layer and patterned to leave openings for the electrodes 24 and bond pads, or openings are created later by means such as laser ablation. Hence the array 10, its supply cable 12 and bond pads are formed of a single body. Additionally, multiple alternating layers of metal and polymer may be applied to obtain more metal traces within a given width.

The pressure applied against cortical neurons, or other neural tissue, by an electrode array is critical. Too little pressure causes increased electrical resistance between the array and cortex, along with electric field dispersion. Too much pressure may block blood flow causing cortical ischemia and hemorrhage. Common flexible circuit fabrication techniques such as photolithography generally require that a flexible circuit electrode array be made flat. Since the cortex is approximately spherical, a flat array will necessarily apply more pressure near its edges, than at its center. Further, the edges of a flexible circuit polymer array may be quite sharp and cut the delicate cortical tissue. With most polymers, it is possible to curve them when heated in a mold. By applying the right amount of heat to a completed array, a curve can be induced that matches the curve of the cortex. With a thermoplastic polymer such as liquid crystal polymer, it may be further advantageous to repeatedly heat the flexible circuit in multiple molds, each with a decreasing radius. Further, it is advantageous to add material along the edges of a flexible circuit array. Particularly, it is advantageous to add material that is more compliant than the polymer used for the flexible circuit. While described as a flexible circuit electrode array, more conventional techniques such as silicone covered wires can also be used.

It is further advantageous to make a thinner polyimide array as the core layer and coat the entire array with a thin layer of Polydimethylsiloxane (PDMS) and to open up the electrode sites and for plating the electrodes with Pt gray or other metal electrode materials. Importantly, a flexible circuit is sufficiently flexible to follow the shape of the cortex.

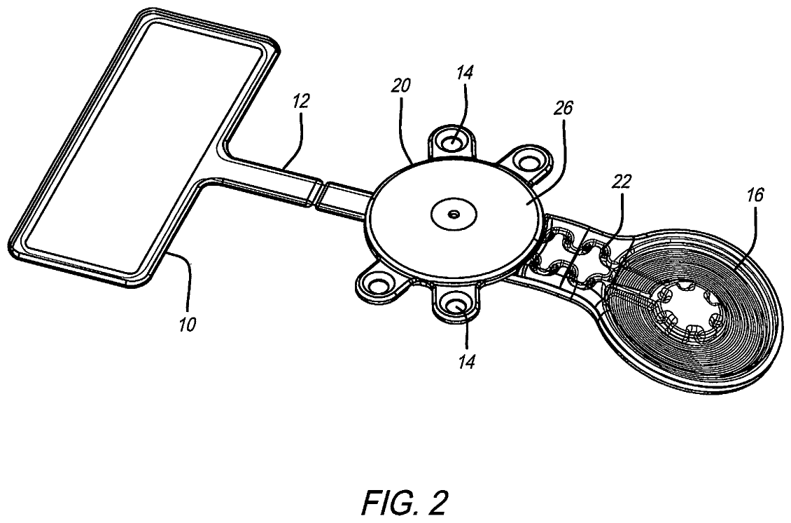

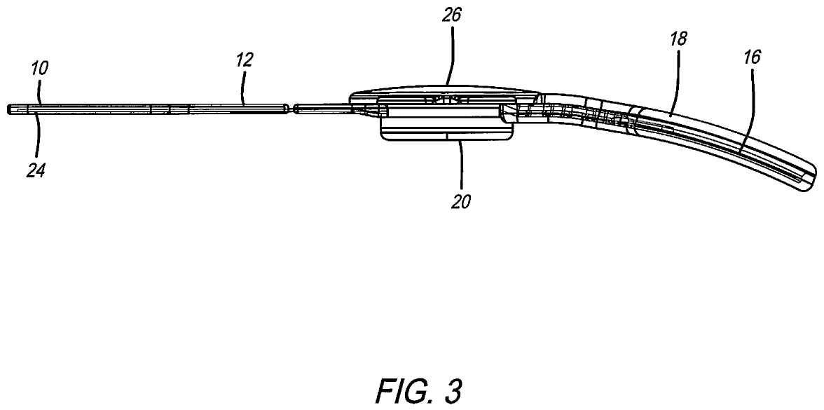

FIG. 2 shows a top perspective view of the implantable cortical stimulator, and FIG. 3 shows a side view of the implantable cortical stimulator. The hermetic package 20 includes a domed lid 24. The domed lid 26 increases impact resistance and reduced irritation of the scalp when the implantable cortical stimulator is implanted. The cortical implant is described in more detail in relation to applicants' cortical visual prosthesis described in U.S. Pat. Nos. 9,861,820 and 9,949,376, which are incorporated herein by reference.

The present invention includes an improved hermetic electronics package for implantation in the human body. A cover is bonded to a substrate having electronic connection vias (manufactured as a “via substrate” using co-fired ceramic technology) such that the cover and via substrate form a hermetic package. In the assembly of hybrid circuits for biomedical devices, surface mount components are often used. The present disclosure includes thin chip components in place of traditional surface mount discrete components. The thin chip components may include stacked capacitors having a low profile, for example, a total height between twenty-five and one hundred-fifty micrometers. The stacked capacitors may be high density trench capacitors and/or metal-on-semiconductor capacitors positioned on an integrated circuit chip. The thin chip components may be a metal-insulator-metal capacitor, wherein the metal-insulator-metal capacitor has a tunable capacitance value and/or is a binary capacitor array. Due to the fact that many biomedical devices are implanted in biological tissues, the use of components with a lower height and reduced lateral footprint is advantageous. Additionally, when using surface mount capacitors to provide tuning capacitance values, it is necessary to prescreen capacitors to be selected for assembly in-process rather than at kitting. The present disclosure includes the advantageous use of components that are tunable in-situ.

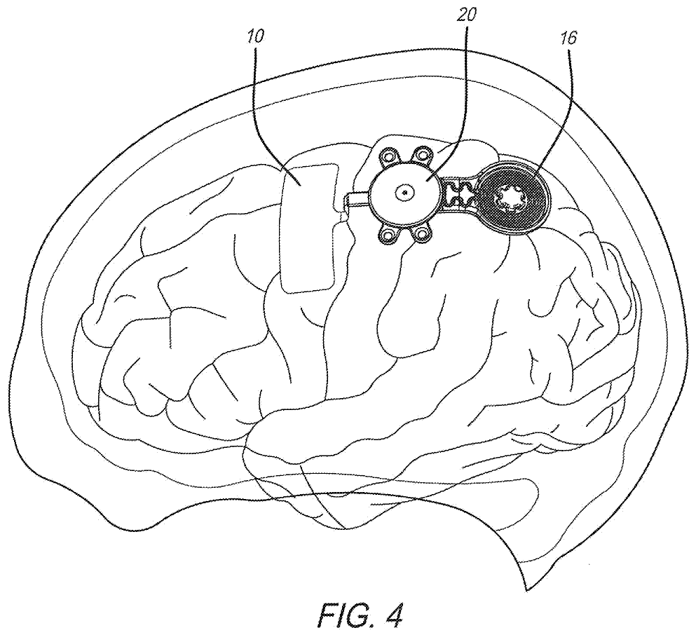

FIG. 4 shows the implantable cortical stimulator and how it is intended to be implanted. The cable 12 pierces the skull and dura to allow the electrode array to be in direct contact with the brain. The hermetic package is placed within an inset burr hole in the skull so it is near flush with the skull. The coil 16 lies on the surface of the skull or within a shallow hollowed out portion. All components, electrode array 10, cable 12 hermetic package 20 and coil 16, remain under the scalp to avoid infection. The most important aspect of successful cortical stimulation for stroke rehabilitation is precise location of stimulation. The present invention includes an electrode array 10 that covers an area slightly larger than the motor cortex, 60 independent electrodes 24, and a 60 channel IPG. This allows a clinician to determine the best location for stimulation in the clinic after surgery is completed.

Therapy may only require one or two channels of active stimulation of a small region of cortex near the array. The advantage of the 60 channel stimulator of the present invention is to allow a clinician to adjust the location of stimulation post surgery, or change the location of stimulation if the initial indication proves incorrect, or provide differing therapy as recovery progresses. The location of stimulation can be adjusted by selecting either a single electrode or groups of electrodes and by using monopolar, bipolar or multipolar configurations. Sequential stimulation of multiple electrodes can be used to target the execution of more complicated movements. It has been established through clinical study that a tonic stimulation over a large area of the motor cortex in conjunction with rehabilitation promotes recovery of function. Such stimulation can be provided by the present invention. The system of the present invention can also provide further therapeutic benefit by targeting stimulation to a desired movement. As an example, in a patient who has having difficulty grasping an object, a clinician can target stimulation to the specific area of the cortex commanding a contraction of muscles responsible for grasping. Repetition of applying the stimulation and the patient attempting to grasp may result in retraining of the neural circuits to improve the patient's ability to grasp independently.

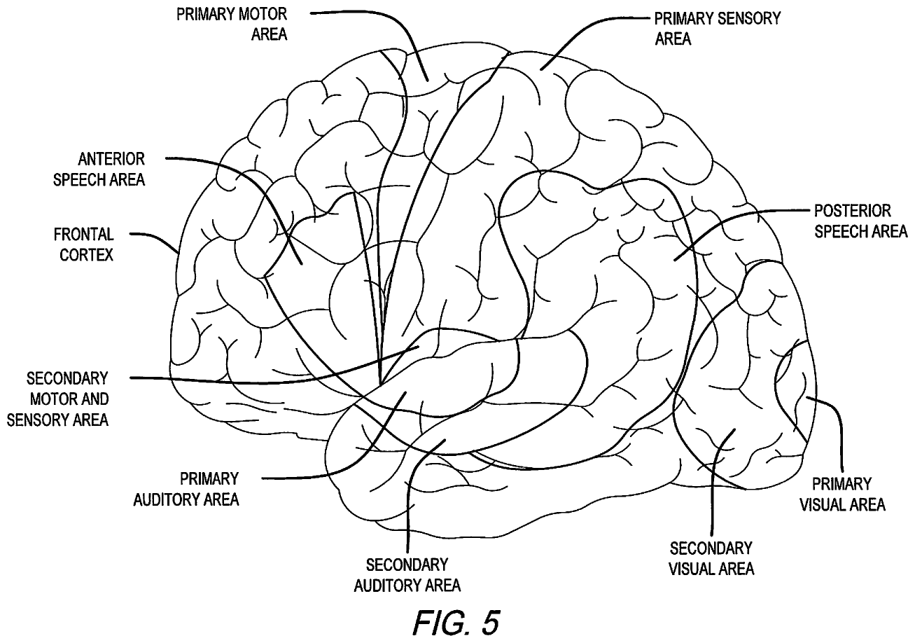

While described as a cortical stimulator for stroke recovery, the present invention is a modification of applicant's cortical stimulator for artificial vision. Referring to FIG. 5, the present invention can be further modified to provide benefit for treating other conditions by modifying the electrode array to cover different portions of the cortex, and providing different control software to provide different stimulation patterns. Examples include stimulating the auditory cortex for tinnitus or deafness, covering the prefrontal cortex for depression or addiction, covering Broca's area for aphasia, covering the eloquent cortex for epilepsy, or covering the motor cortex for traumatic brain injury. In each case, the 60 channel stimulator of the present invention allows for covering the entire target area and fine tuning the location of stimulation by clinical experimentation post surgery.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art as a complete disclosure and description of how to make and use the embodiments of the disclosure, and are not intended to limit the scope of what the inventor/inventors regard as their disclosure.

Modifications of the above-described modes for carrying out the methods and systems herein disclosed that are obvious to persons of skill in the art are intended to be within the scope of the following claims. It is to be understood that the disclosure is not limited to particular methods or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

What is claimed is:

1. A neurostimulator to support stroke rehabilitation comprising:
an external portion including
a processor adapted to generate stimulation patterns and select drivers for those stimulation patterns, the processor driving an external coil, the external coil providing power and data, including the stimulation patterns and selected drivers, to an implanted coil;
an implanted portion including
an implanted coil receiving power and data from the external coil and providing power and data to an electronic circuit within a hermetic package;
the electronic circuit including a plurality of drivers, each driver electrically connected to one of a plurality of electrodes,
an electrode array adapted to cover a substantial portion of motor cortex of a brain, including the plurality of electrodes, each electrode covering a portion of the motor cortex of the brain.

2. The neurostimulator according to claim 1, wherein the driver selection is based on visual observation of muscle twitch feedback.

3. the neurostimulator according to claim 1, wherein the muscle twitch feedback is determined by measuring motor evoked potentials.

4. The neurostimulator according to claim 1, wherein the substantial portion of the motor cortex is the upper extremity region of the motor cortex.

5. The neurostimulator according to claim 1, wherein the electrode array is adapted to cover more than the motor cortex to ensure full coverage of the motor cortex.

6. the neurostimulator according to claim 1, wherein the electrode array is a flexible circuit.

7. The neurostimulator according to claim 6, wherein the flexible circuit is a sandwich of polyimide layers with platinum traces, and over molded with Polydimethylsiloxane.

8. A neurostimulator:
an external portion including
a processor adapted to generate stimulation patterns and select drivers for those stimulation patterns, the processor driving an external coil, the external coil providing power and data, including the stimulation patterns and selected drivers, to an implanted coil;
an implanted portion including
an implanted coil receiving power and data from the external coil and providing power and data to an electronic circuit within a hermetic package;
the electronic circuit including a plurality of drivers, each driver electrically connected to one of a plurality of electrodes,
an electrode array adapted to cover a portion of the cortex of a brain, including the plurality of electrodes, each electrode covering a portion of the cortex of the brain.

9. The neurostimulator according to claim 8, wherein the electrode array is adapted to cover the prefrontal cortex.

10. The neurostimulator according to claim 8, wherein the electrode array is adapted to cover the auditory cortex.

11. The neurostimulator according to claim 8, wherein the electrode array is adapted to cover the eloquent cortex.

12. The neurostimulator according to claim 8, wherein the electrode array is adapted to cover the speech cortex.

13. the neurostimulator according to claim 8, wherein the electrode array is a flexible circuit.

14. The neurostimulator according to claim 13, wherein the flexible circuit is a sandwich of polyimide layers with platinum traces, and over molded with Polydimethylsiloxane.
